(19) United States
(12) Patent Application Publication
(10) Pub. No.: US 2024/0167010 A9
(43) Pub. Date: May 23, 2024

(54) A GENETICALLY MODIFIED CELL LINE PRODUCING A RECOMBINANT GLYCOPROTEIN HAVING A MANNOSE-TERMINATED N-GLYCAN

(71) Applicant: WUXI BIOLOGICS IRELAND LIMITED, CO LOUTH (IE)

(72) Inventors: Liqin XU, Shanghai (CN); Fenglin WANG, Shanghai (CN); Yangfang ZHOU, Shanghai (CN); Yangzi PENG, Shanghai (CN); Jiexing CAI, Shanghai (CN); Weichang ZHOU, Shanghai (CN); Xiaobo XI, Shanghai (CN); Weiwen WANG, Shanghai (CN); Tingting WANG, Shanghai (CN); Chunsheng YANG, Shanghai (CN); Quanmin CHEN, Shanghai (CN); Jeremy GUO, Shanghai (CN)

(73) Assignee: WUXI BIOLOGICS IRELAND LIMITED, CO LOUTH (IE)

(21) Appl. No.: 17/996,441

(22) PCT Filed: Apr. 21, 2021
(86) PCT No.: PCT/CN2021/088609

§ 371 Date: Oct. 18, 2022

(30) Foreign Application Priority Data
Apr. 23, 2020 (WO) PCT/CN2020/086499
Apr. 16, 2021 (CN) 202110411715.6

(51) Int. Cl. C12N 9/24; A61K 38/47; C12N 9/10; C12N 15/90

(52) U.S. Cl. CPC C12N 9/2402; A61K 38/47; C12N 9/1051; C12N 15/907; C12Y 204/01101; C12Y 302/01045; C12N 2310/20

(57) ABSTRACT

Provided are a genetically modified cell line producing a recombinant glycoprotein having a mannose-terminated N-glycan and a method for producing the recombinant glycoprotein or a method for generating the cell, clone or cell line. The cell line comprises an insertion of less than 600 bp in the coding region of a chromosomal sequence encoding MGAT1.

### FIELD OF THE INVENTION

This application generally relates to a genetically modified cell line producing a recombinant glycoprotein having a mannose-terminated N-glycan and a method for producing the recombinant glycoprotein or a method for generating the cell, clone or cell line.

### BACKGROUND OF THE INVENTION

One of commercially useful glycoproteins is β-glucocerebrosidase which catalyzes the hydrolysis of glucocerebroside into ceramide and glucose. Since an in vivo deficiency in β-glucocerebrosidase, which causes the accumulation of glucoceramide within peripheral macrophages, resulting in cellular enlargements, may cause Gaucher's disease which is an autosomal recessive lysosomal storage disease resulting in malfunction and deterioration of spleen, liver and bone marrow, exogenous administration of recombinantly expressed β-glucocerebrosidase is considered as an attractive way to treat the Gaucher's disease in the art.

A problem is that, an unmodified human β-glucocerebrosidase derived from placenta can hardly be taken up by cells in need, such as dendritic cells and macrophages, through mannose receptor-mediated endocytosis due to lack of exposed mannose residues. Accordingly, there are increasing demands for a convenient and effective way to produce a recombinantly expressed glycoprotein, e.g. β-glucocerebrosidase, having a mannose-terminated N-glycan, such as by removing the residues which block the mannose in the N-glycan (exposing the mannose as the terminal residue of the N-glycan), for facilitating mannose receptor-mediated uptake of the glycoproteins by cells in need.

Therefore, there is a need for cell lines that are engineered to produce recombinant glycoproteins with mannose-terminated N-glycan. Moreover, it is desirable that these engineered cell lines stably produce proteins with terminal mannose residues, while retaining high protein productivity, good quality, and high enzyme activity.

### SUMMARY OF THE INVENTION

The inventors of the present disclosure had surprisingly discovered a genetically modified cell line deficient in mannosyl (alpha-1,3-)-recombinant glycoprotein beta-1,2-N-acetylglucosaminyltransferase 1 (MGAT1) gene by insertion mutation, wherein the cell line comprises an insertion of less than 600 bp in the coding region of the chromosomal sequence encoding MGAT1 , by selection with RCA. The recombinant glycoprotein produced by the cell line in the invention has high protein productivity, high protein quality, and high enzyme activity.

Among the various aspects of the present disclosure is the provision of a cell line deficient in mannosyl (alpha-1,3-)-glycoprotein beta-1,2-N-acetylglucosaminyltransferase 1 (MGAT1), wherein the cell line comprises an insertion of less than 600 bp in the coding region of the chromosomal sequence encoding MGAT1. In one embodiment, the cell line comprises an insertion of less than 400, 200, 100, 50, 30, 10, or 5 bp in the coding region of a chromosomal sequence encoding MGAT1 . In one embodiment, the cell line comprises an insertion of 1 bp in the coding region of a chromosomal sequence encoding MGAT1. In one embodiment, the chromosomal sequence encoding MGAT1 is modified using a targeting endonuclease-mediated genome editing technology, for example, targeting endonuclease-mediated genome editing technology is a Clustered Regularly Interspaced Short Palindromic Repeats (CRISPR), Zinc Finger nuclease (ZFN) or Transcription activator-like effector nuclease (TALEN). In one embodiment, the cell is an animal cell, preferably a mammalian cell, further preferably a Chinese Hamster Ovary (CHO) cell. In one embodiment, the cell line expresses at least one glycoprotein comprising one or more terminal mannose residues. In one embodiment, glycoprotein is an enzyme, such as β-glucocerebrosidase.

Another aspect of the disclosure encompasses a method for producing a recombinant glycoprotein comprising:
(1) culturing the genetically modified cell line; and
(2) recovering the glycoprotein.

Another aspect of the disclosure encompasses a method for producing a recombinant glycoprotein having one or more terminal mannose residues having a mannose-terminated N-glycan comprising:
(1) introducing a gene encoding the recombinant protein into a cell line,
(2) mutating mannosyl (alpha-1,3-)-recombinant glycoprotein beta-1,2-N-acetylglucosaminyltransferase 1 (MGAT1) gene in the cell line obtained by step (1); and
(3) expressing the recombinant glycoprotein;
wherein, the MGAT1 gene is mutated by CRISPR, in which sgRNA comprises or consists of a nucleic acid sequence of SEQ ID NO: 4, or comprises or consists of a nucleic acid sequence having at least 90%, 95%, 96%, 97%, 98% or 99% sequence identity to SEQ ID NO: 4.

In one embodiment, the cell line is selected with RCA. In one embodiment, the recombinant glycoprotein is an enzyme, such as β-glucocerebrosidase. In one embodiment, the method further comprises a step of selecting the mutated cell line in a batch refeeding assay. In one embodiment, the batch refeeding assay comprises a step of refreshing the production medium daily. In one embodiment, the recombinant glycoprotein has a N-glycan containing one or more terminal mannose residues, preferably 1 to 9 terminal mannose residues, more preferably 3 to 6 terminal mannose residues, and optionally 1 to 3 fucose residues which do not block the terminal mannose residues, wherein the N-glycan is selected from the group consisting of Man3, Man4, Man4+1F, Man5, Man5+1F, and Man6. In one embodiment, the proportion of the mannose-terminated N-glycans attached to the recombinantly glycoprotein is more than 80%, 85%, 86%, 87%, 88%, or 89%. In one embodiment, the enzyme activity is more than 0.5 U/mL. In one embodiment, the cell is an animal cell, preferably a mammalian cell, further preferably a Chinese Hamster Ovary (CHO) cell.

Another aspect of the disclosure encompasses a recombinant glycoprotein, such as β-glucocerebrosidase, produced by the cell line or the method in the disclosure. Another aspect of the disclosure encompasses a method for treating a β-glucocerebrosidase-deficiency-related disease, such as Gaucher's disease, comprising administering the recombinant glycoprotein such as β-glucocerebrosidase in the disclosure to a subject in need thereof. Still another aspect of the present disclosure is a cell, clone or cell line generated by the method.

Another aspect of the present disclosure is a pharmaceutical formulation comprising a therapeutically effective amount of a recombinant enzyme, a buffer, an osmoregulator and a surfactant. In one embodiment, the enzyme is β-glucocerebrosidase present an amount of 50-150 U/ml. In one embodiment, the buffer is a citrate buffer present at an amount of 2-10 g/mL. In one embodiment, the osmoregulator is sucrose present at amount of 40-120 g/L. In one embodiment, the surfactant is polysorbate 80 present at an amount of 0.1-1 g/L. In one embodiment, the formulation has a pH of 5-7.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Further, the contents of all references, patents and published patent applications cited throughout this application are incorporated herein in entirety by reference.

### BRIEF DESCRIPTION OF THE FIGURES

FIG. 1 is the plasmid containing Cas 9 and sgRNA_5.

FIG. 2 is a glycan chromatogram of the monoclone in which MGAT1 gene had been mutated.

FIG. 3 is a glycan chromatogram of the control pool with wild type MGAT1.

### DETAILED DESCRIPTION OF THE INVENTION

While the present disclosure may be embodied in many different forms, disclosed herein are specific illustrative embodiments thereof that exemplify the principles of the present disclosure. It should be emphasized that the present disclosure is not limited to the specific embodiments illustrated. Moreover, any section headings used herein are for organizational purposes only and are not to be construed as limiting the subject matter described.

### Definitions

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised,” is not limiting. In addition, ranges provided in the specification and appended claims include both end points and all points between the end points.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

In the context of the present disclosure, MGAT1 , which is also known as N-acetylglucosaminyltransferase I (GlcAc-T I or GnT I) or N-glycosyl-oligosaccharide-glycoprotein N-acetylglucosaminyltransferase I, catalyzes the transfer of N-acetylglucosamine (GlcNAc) onto the oligomannose core (i.e., Man5GlcNAc2 (Man5) moiety) of a growing N-glycan. Malfunction of the MGAT1 will affect the N-glycan profile, such that a glycoprotein, e.g. β-glucocerebrosidase, having a N-glycan with terminal mannose residues can be obtained. In the context of the present disclosure, the MGAT1 is used interchangeably with the GnT I.

The term “monoclone” as used herein refers to a group of cells produced from a single ancestral cell by repeated cellular replication. In a more specific context of the present disclosure, a monoclone expressing a recombinant glycoprotein, such as β-glucocerebrosidase, may be generated by cloning a single cell derived from a pool expressing the recombinant glycoprotein, such as β-glucocerebrosidase.

The term “mutation” or “mutating” or “mutated” as used herein refers to deletion or insertion mutation. In a more specific context of the present disclosure, insertion mutation means insertion of 1, 2, 3, 4, 5, 6, 7, 8, 9, 10, or more base pairs in the gene coding the target protein or in the regulatory nucleic acid sequence, e.g., promoter or enhancer, operably coupled to the gene encoding the protein. In the specific Example, mutating mannosyl (alpha-1,3-)-recombinant glycoprotein beta-1,2-N-acetylglucosaminyltransferase 1 (MGAT1) gene in the cell line is performed by insertion mutation. Deletion mutation means deletion of 1, 2, 3, 4, 5, 6, 7, 8, 9, 10, or more base pairs in the gene coding the target protein or in the regulatory nucleic acid sequence, e.g., promoter or enhancer, operably coupled to the gene encoding the protein. The skilled in the art knows that knocking out comprises introducing a deletion, substitution, or insertion mutation to the gene encoding the target protein or to the regulatory nucleic acid sequence, e.g., promoter or enhancer operably coupled to the gene encoding the protein, wherein the mutation reduces the level of expression or functional deficiency e.g. inactivation of the protein.

The term “pool” as used herein refers to a mixed cell population. In a more specific context of the present disclosure, a pool expressing a recombinant glycoprotein, such as β-glucocerebrosidase, may be generated by transfecting a gene encoding the recombinant glycoprotein, such as β-glucocerebrosidase, to a host cell.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I, U) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. In the present disclosure, the percentage of sequence identity may be at least 80%, 81%, 82%, 83%, 84%, 85%, 86%, 87%, 88%, 89%, 90%, 91%, 92%, 93%, 94%, 95%, 96%, 97%, 98%, 99%, or 100%.

The term “pharmaceutical formulation” refers to a formulation that is in a form such as to allow the biological activity of the active ingredient to be effective, and that does not contain additional components that have unacceptable toxicity to the subject to which the formulation is to be administered.

The term “stable” used in the context of a pharmaceutical formulation refers to a formulation in which the protein retains its physical stability and/or chemical stability and/or biological activity upon storage. Stability can be measured at a selected temperature for a selected time period. Preferably, the formulation is stable at room temperature (about 25° C.) or at 40° C. for at least 3 months and/or stable at about 2-8° C. for at least 1 year.

The term “buffer” refers to a buffered solution that resists changes in pH by the action of its acid-base conjugate components. The buffer of this invention has a pH in the range from about 4 to about 8; preferably from about 4.5 to about 7; and most preferably has a pH in the range from about 5.0 to about 6.5. Examples of buffers that will control the pH in this range include acetate, succinate, gluconate, histidine, citrate and other organic acid buffers.

The term “osmoregulator” refers to an agent added to the formulation so as to achieve desired level of osmolality. The osmolality can be expressed as the concentration of osmotically active particles dissolved in 1 kg of solution. Examples of osmoregulator include sugars and/or sugar alcohol.

The term “surfactant” refers to a surface-active agent. Examples of surfactants used herein include polysorbate and poloxamer.

The term “reconstitution” refers to dissolving a lyophilized protein formulation in a diluent such that the protein is dispersed therein. The reconstituted formulation is suitable for administration.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. The nomenclature used in connection with, and the laboratory procedures and techniques of, analytical chemistry, synthetic organic chemistry, and medicinal and pharmaceutical chemistry described herein are those well-known and commonly used in the art. Moreover, any section headings used herein are for organizational purposes only and are not to be construed as limiting the subject matter described.

### Cell Line

One aspect of the present disclosure encompasses a cell line deficient in mannosyl (alpha-1,3-)-glycoprotein beta-1,2-N-acetylglucosaminyltransferase 1 (MGAT1), wherein the cell line comprises an insertion of less than 600 bp in the coding region of the chromosomal sequence encoding MGAT1.

In one embodiment, the MGAT1 deficient cell line comprises an insertion ranging from about 1 bp to 599 bp in the chromosomal sequence encoding MGAT1 . In another embodiment, the MGAT1 deficient cell line comprises an insertion ranging from about 1 bp to about 50 bp, from about 50 bp to about 100 bp, from about 100 bp to about 200 bp, from about 200 bp to about 300 bp, from about 300 bp to about 400 bp, or less than about 600 bp in coding region of MGAT1 . In exemplary embodiments, the MGAT1 deficient cell line comprises an insertion of 1 bp, 2 bp, 3 bp, 4 bp, 5 bp, 10 bp, 25 bp, or 50 bp in the chromosomal sequence encoding MGAT1, which results in the functional deficiency of MGAT1. An insertion ranging from about 1 bp to 599 bp in the chromosomal sequence encoding MGAT1 results in a loss or inactivation of MGAT1 expression. The MGAT1 coding sequence undergoes a shift in the reading frame, thereby preventing production of a protein product. The chromosomal sequence encoding MGAT1 can be mutated using targeting endonuclease-mediated genome editing technology as detailed below.

In one embodiment, the MGAT1 deficient cell line comprises a deletion ranging from about 1 bp to 1300 bp in the chromosomal sequence encoding MGAT1. In another embodiment, the MGAT1 deficient cell line comprises a deletion ranging from about 1 bp to about 50 bp, from about 50 bp to about 100 bp, from about 100 bp to about 200 bp, from about 200 bp to about 300 bp, from about 300 bp to about 400 bp, 400 bp-600 bp, or more than about 600 bp in coding region of MGAT1. In exemplary embodiments, the MGAT1 deficient cell line comprises a deletion of 1 bp, 2 bp, 3 bp, 4 bp, 5 bp, 10 bp, 25 bp, or 50 bp in the chromosomal sequence encoding MGAT1 , which results in the functional deficiency of MGAT1. A deletion ranging from about 1 bp to 599 bp in the chromosomal sequence encoding MGAT1 results in a loss or inactivation of MGAT1 expression. The MGAT1 coding sequence undergoes a shift in the reading frame, thereby preventing production of a protein product. The chromosomal sequence encoding MGAT1 can be mutated using targeting endonuclease-mediated genome editing technology as detailed below.

In some embodiments, the cells which can be used to produce the recombinant glycoprotein are in principle all cells known to the person skilled in the art, which have the ability to express the recombinant glycoprotein. The cells may be animal cells, in particular mammalian cells. Examples of mammalian cells include CHO (Chinese Hamster Ovary) cells, preferably CHO-K1 cells, hybridomas, BHK (Baby Hamster Kidney) cells, myeloma cells, human cells, for example HEK-293 cells, human lymphoblastoid cells, E1 immortalized HER cells, mouse cells, for example NSO cells. In one embodiment, the cell is an animal cell, preferably a mammalian cell, further preferably a Chinese Hamster Ovary (CHO) cell. In exemplary embodiments, the cell line is a type that is widely used for the production of recombinant proteins, such as glycoproteins, and the like. In exemplary embodiments, the cell line is a CHO cell line. Numerous CHO cell lines are available from ATCC. Suitable CHO cell lines include, but are not limited to, CHO-K1 cells and derivatives thereof.

In one embodiment, the cell line expresses at least one glycoprotein comprising one or more terminal mannose residues. In one embodiment, glycoprotein is β-glucocerebrosidase.

### Targeting Endonuclease-Mediated Genome Editing Technology

In one embodiment, the chromosomal sequence encoding MGAT1 is modified using a targeting endonuclease-mediated genome editing technology, for example, targeting endonuclease-mediated genome editing technology is a Clustered Regularly Interspaced Short Palindromic Repeats (CRISPR), Zinc Finger nuclease (ZFN) or Transcription activator-like effector nuclease (TALEN).

CRISPR systems, which form an adaptive immune system in bacteria, have been modified for genome engineering. Engineered CRISPR systems contain two components: a guide RNA (gRNA or sgRNA) and a CRISPR-associated endonuclease (Cas endonuclease). The gRNA is a short synthetic RNA composed of a scaffold sequence necessary for Cas-binding and a user-defined ˜20 nucleotide spacer that defines the genomic target to be modified. In the present disclosure, the gRNA and sgRNA can be used interchangeably with each other. Thus, one can change the genomic target of the Cas protein by simply changing the target sequence present in the gRNA.

One can use CRISPR to generate knockout e.g. mutated cells or animals by co-expressing an endonuclease like Cas9 or Cpf1 and a gRNA specific to the gene to be targeted. The genomic target can be any ˜20 nucleotide DNA sequence, provided it meets two conditions: (1) the sequence is unique compared to the rest of the genome; and (2) the target is present immediately adjacent to a Protospacer Adjacent Motif (PAM). The PAM sequence is essential for target binding, but the exact sequence depends on which Cas protein is used. A popular Cas protein is Streptococcus pyogenes Cas9 (SpCas9). Once expressed, the Cas9 protein and the gRNA form a ribonucleoprotein complex through interactions between the gRNA scaffold and surface-exposed positively-charged grooves on Cas9. Cas9 undergoes a conformational change upon gRNA binding that shifts the molecule from an inactive, non-DNA binding conformation into an active DNA-binding conformation. Importantly, the spacer region of the gRNA remains free to interact with target DNA.

Cas9 will only cleave a given locus if the gRNA spacer sequence shares sufficient homology with the target DNA. Once the Cas9-gRNA complex binds a putative DNA target, the seed sequence (8-10 bases at the 3′ end of the gRNA targeting sequence) will begin to anneal to the target DNA. If the seed and target DNA sequences match, the gRNA will continue to anneal to the target DNA in a 3′ to 5′ direction. The zipper-like annealing mechanics of Cas9 may explain why mismatches between the target sequence in the 3′ seed sequence completely abolish target cleavage, whereas mismatches toward the 5′ end distal to the PAM often still permit target cleavage.

The Cas9 nuclease has two functional endonuclease domains: RuvC and HNH. Cas9 undergoes a second conformational change upon target binding that positions the nuclease domains to cleave opposite strands of the target DNA. The end result of Cas9-mediated DNA cleavage is a double-strand break (DSB) within the target DNA (˜3-4 nucleotides upstream of the PAM sequence).

The resulting DSB is then repaired by one of two general repair pathways: (1) the efficient but error-prone non-homologous end joining (NHEJ) pathway; and (2) the less efficient but high-fidelity homology directed repair (HDR) pathway. The NHEJ repair pathway is the most active repair mechanism, and it frequently causes small nucleotide insertions or deletions (indels) at the DSB site. The randomness of NHEJ-mediated DSB repair has important practical implications, because a population of cells expressing Cas9 and a gRNA will result in a diverse array of mutations. In most cases, NHEJ gives rise to small indels in the target DNA that result in amino acid deletions, insertions, or frameshift mutations leading to premature stop codons within the open reading frame (ORF) of the targeted gene. The ideal end result is a loss-of-function mutation within the targeted gene.

In a specific embodiment, knockout mutation of the MGAT1 gene by means based on CRISPR can be carried out by any protocol feasible, for example, but not limited to that taught in Yang 2014 (Yang, et al., CRISPR/Cas9-Directed Genome Editing of Cultured Cells. Current Protocols in Molecular Biology, 2014, 107(1):31.1.1-31.1.17). In a more specific embodiment, the MGAT1 gene is mutated by means based on CRISPR, in which the sgRNA comprises or consists of SEQ ID NO: 4, or comprises or consists of a nucleic acid sequence having at least 90%, 95%, 96%, 97%, 98% or 99% sequence identity to SEQ ID NO: 4.

### Method for Producing a Recombinant Glycoprotein, a Cell, Clone or Cell Line

Another aspect of the present disclosure encompasses a method for producing a recombinant glycoprotein having one or more terminal mannose residues having a mannose-terminated N-glycan comprising:
(1) introducing a gene encoding the recombinant protein into a cell line;
(2) mutating mannosyl (alpha-1,3-)-recombinant glycoprotein beta-1,2-N-acetylglucosaminyltransferase 1 (MGAT1) gene in the cell line obtained by step (1); and
(3) expressing the recombinant glycoprotein;
wherein, the MGAT1 gene is mutated by CRISPR, in which sgRNA comprises or consists of a nucleic acid sequence of SEQ ID NO: 4, or comprises or consists of a nucleic acid sequence having at least 90%, 95%, 96%, 97%, 98% or 99% sequence identity to SEQ ID NO: 4.

A further aspect of the present disclosure encompasses a method for generating a cell, clone or cell line producing a recombinant glycoprotein having a mannose-terminated N-glycan comprising:
(1) introducing a gene encoding the recombinant protein into a cell line;
(2) mutating mannosyl (alpha-1,3-)-recombinant glycoprotein beta-1,2-N-acetylglucosaminyltransferase 1 (MGAT1) gene in the cell line obtained by step (1); and
wherein, the MGAT1 gene is mutated by CRISPR, in which sgRNA comprises or consists of a nucleic acid sequence of SEQ ID NO: 4, or comprises or consists of a nucleic acid sequence having at least 90%, 95%, 96%, 97%, 98% or 99% sequence identity to SEQ ID NO: 4.

The plasmids usable in the present disclosure can be any one commercially available for the intended purposes. The skilled artisan will readily know which plasmids are suitable for which purposes and would appreciate that variances in the plasmids used will not substantially vary the technical effects of the methods/products of the present disclosure.

In one embodiment, a plasmid for introducing a gene into a cell, such as CHO cell, can be prepared manually by integrating elements, such as a Replication Origin (ori), a promoter, a multicloning site for transferring gene of interest, and one or more selection markers-encoding genes, into a single circular sequence. A non-limiting example of a conventionally usable plasmid for introducing a gene into a cell, such as CHO cell, may be pMX241 (Addgene Cat. No. 23017) or the like.

In one embodiment, a plasmid by means of CRISPR can be prepared manually by integrating elements, such as a Replication Origin (ori), a promoter, a sgRNA scaffold for accepting an intendedly designed sgRNA, a gene encoding CRISPR-associated (Cas) endonuclease, and one or more selection markers-encoding genes, into a single circular sequence. A non-limiting example of a conventionally usable knockout vector by means of CRISPR may be pSpCas9(BB)-2A-Puro (PX459) V2.0 (Addgene Cat. No. 62988) or the like.

In the present disclosure, the selection marker can be any one commercially available in the art. For example, the selection marker comprises, but not limited to, Neomycin, Puromycin, Hygromycin, Zeocin, Blasticidin, or the like.

In one embodiment, the cell line is selected with RCA. In one embodiment, the transfected cell is incubated with Ricinus communis agglutinin-I (RCA-I). The MGAT1 deficient cell lines can be further enriched by incubation with Ricinus communis agglutinin-I (RCA-I). RCA-I is a cytotoxic lectin that does not bind terminal mannose residues and therefore allows for the selection of cells devoid of MGAT1 activity because such cells produce glycoproteins with terminal mannose residues.

In one embodiment, the cell is an animal cell, preferably a mammalian cell, further preferably a Chinese Hamster Ovary (CHO) cell. In exemplary embodiments, the cell line is a type that is widely used for the production of recombinant proteins, such as glycoproteins, and the like. In exemplary embodiments, the cell line is a CHO cell line. Numerous CHO cell lines are available from ATCC. Suitable CHO cell lines include, but are not limited to, CHO-K1 cells and derivatives thereof.

### Recombinant Glycoprotein

A further aspect of the present disclosure encompasses a recombinant glycoprotein, e.g. β-glucocerebrosidase, having a mannose-terminated N-glycan produced by the method of the present disclosure.

In one embodiment, the recombinant glycoprotein is an enzyme, such as β-glucocerebrosidase. In one embodiment, the recombinant glycoprotein has a N-glycan containing one or more terminal mannose residues. In some embodiments, the recombinant glycoprotein has a N-glycan containing 2 to 9 terminal mannose residues (called also as Man2 to Man9), such as 3 to 6 terminal mannose residues (called also as Man3 to Man6), and optionally 1 to 3 fucose residues (called also as 1F to 3F), such as 1 fucose residue (called also as 1F) which do not block the terminal mannose residues. An exemplary N-glycan in the present disclosure is selected from the group consisting of Man3, Man4, Man4+1F, Man5, Man5+1F, Man6, etc. In one embodiment, the proportion of the mannose-terminated N-glycans attached to the recombinantly glycoprotein is more than 80%, 85%, 86%, 87%, 88%, or 89%.

Enzyme activity was measured using p-nitrophenyl-b-D-glucopyranoside (Sigma-N7006) as a substrate. The released product was 4-Nitrophenol (Sigma-35836-1G) which was detected by measuring the absorbance at 405 nm using a plate reader (MOLECULAR DEVICE, i3X). A reference standard curve was assayed in parallel to quantitate concentrations of 4-Nitrophenol. Enzyme activity is defined as the amount of enzyme that catalyzes 1 nanomole of 4-Nitrophenol released from p-nitrophenol β-D-glucopyranoside per mL per hour at 37° C. In one embodiment, the enzyme activity β-glucocerebrosidase produced by the cell line in the disclosure is more than about 0.2 U/mL, about 0.3 U/m, about 0.4 U/mL, about 0.5 U/mL, about 0.6 U/mL.

The recombinant glycoprotein was purified and the purity is more than about 70%, more than about 75%, more than about 80%, more than about 85%, more than about 90%. The purity can be detected by Caliper-SDS and SEC-HPLC assay, respectively.

### Method for Treating

A further aspect of the present disclosure encompasses a method for treating a β-glucocerebrosidase-deficiency-related disease, such as Gaucher's disease, comprising administering a recombinant glycoprotein, e.g. β-glucocerebrosidase, having a mannose-terminated N-glycan of the present disclosure to a subject in need thereof, or encompasses use of a recombinant glycoprotein, e.g. β-glucocerebrosidase, having a mannose-terminated N-glycan of the present disclosure in the manufacture of a composition for treating a β-glucocerebrosidase-deficiency-related disease, such as Gaucher's disease.

### Technical Effect of the Present Disclosure

The technical effects attained by the method/product of the present disclosure can be summarized as follows.

First, by mutation of the MGAT1 gene in a cell, such as CHO cell, which expresses a recombinant glycoprotein, such as β-glucocerebrosidase, the present disclosure has high purity good quality, and increased enzyme activity.

Second, the present disclosure has developed new plasmid comprising specific sgRNA with higher mutation efficiency (e.g. sgRNA_5 as set forth in SEQ ID NO: 4).

Third, in the present disclosure, the MGAT1 deficient cell lines can be further enriched by incubation with Ricinus communis agglutinin-I (RCA-I) in the steps of producing the MGAT1 deficient cell lines.

Fourth, in the present disclosure, the gene encoding the recombinant glycoprotein is introduced into a cell prior to the mutation of the MGAT1 gene in the cell, beneficial from which, a recombinant glycoprotein having a mannose-terminated N-glycan could be produced in a more time-, cost- and labor-effective manner than the prior art methods due to clear Product Quality Attributes (PQA) and productivity requirement on the expressed recombinant glycoprotein for clone screening. By contrast, screening of best-performed host cell with MGAT1 gene mutation and then introduce the gene encoding the recombinant glycoprotein into mutated host cell would become much more difficult as there is no such clearly defined criteria on PQA and productivity for screening the best host cell. It will take shorter time to screen a better clone than a host cell. Additionally, the mutation and cloning are carried out in the same step in the method of the present disclosure.

### Pharmaceutical Formulation

A further aspect of the present disclosure encompasses a pharmaceutical formulation of β-glucocerebrosidase. The formulation comprises a therapeutically effective amount of β-glucocerebrosidase, a buffer, an osmoregulator and a surfactant. In one embodiment, β-glucocerebrosidase is present an amount of 50-150 U/ml, 75-125 U/ml, 80-120 U/ml, 90-110 U/ml or 100 U/ml. In one embodiment, the buffer is a citrate buffer present at an amount of 2-10 g/mL, 3-9 g/mL., 4-8 g/mL, 5-7 g/mL, or 5-6 g/mL. In one embodiment, the osmoregulator is sucrose present at amount of 40-120 g/L, 50-110 g/L, 60-100 g/L, 70-90 g/L or 80 g/L. In one embodiment, the surfactant is polysorbate 80 present at an amount of 0.1-1 g/L, 0.1-0.9 g/L, 0.1-0.8 g/L, 0.1-0.7 g/L, 0.1-0.6 g/L, 0.1-0.5 g/L, 0.1-0.4 g/L, 0.1-0.3 g/L, or 0.2 g/L. In one embodiment the formulation has a pH of 5-7.

The pharmaceutical formulation described in the present disclosure provides a stable formulation of β-glucocerebrosidase with extended shelf life and enhanced stability. The formulation is stable at 2-8° C. for at least 12 weeks or at room temperature (about 25° C.) for at least 12 weeks or at 40° C. for at least 4 weeks. The formulation retains its physical and/or chemical and/or biological activity upon storage under different conditions.

### Sequence Listing Summary

Appended to the instant application is a sequence listing comprising a number of nucleic acid and amino acid sequences as follows.
SEQ ID NO: 1 is an amino acid sequence of human β-glucocerebrosidase (NP_000148.2) (https://www.ncbi.nlm.nih.gov/protein/NP_000148.2).
SEQ ID NO: 2 is a nucleic acid sequence of human β-glucocerebrosidase (NM_000157.3) (https://www.ncbi.nlm.nih.gov/nuccore/NM_000157.3).
SEQ ID NO: 3 is a genomic nucleic acid sequence of MGAT1 gene in CHO-K1 cell.
SEQ ID NO: 4 is a nucleic acid sequence of sgRNA_5.

### Abbreviations

Cas: CRISPR-associated
CEX: Cation Exchange
CHO cells: Chinese Hamster Ovary cells
CRISPR: Clustered Regularly Interspaced Short Palindromic Repeats
DSB: double-strand break
FACS: Fluorescence activated Cell Sorting
GnT I: N-Acetyl-glucosaminyltransferase I
gRNA: guide RNA
HDR: homology directed repair
HILIC: hydrophilic interaction liquid chromatography
LDC: Limiting Dilution Cloning
MGAT1: mannosyl (alpha-1,3-)-recombinant glycoprotein beta-1,2-N-acetylglucosaminyltransferase
NHEJ: non-homologous end joining
ORF: open reading frame
ori: Replication Origin
PAM: Protospacer Adjacent Motif
PCR: Polymerase Chain Reaction
Qp: Specific Productivity
RT: Response versus Acquisition Time
sgRNA: single-guide RNA
SpCas9: Streptococcus pyogenes Cas9
TALEN: Transcription activator-like effector nuclease
UPLC: ultra performance liquid chromatography
VCD: variable cell density
VIA: Viability
ZFN: Zinc Finger nuclease
2-AB: 2-aminobenzamide

### EXAMPLES

The present disclosure, thus generally described, will be understood more readily by reference to the following Examples, which are provided by way of illustration and are not intended to be limiting of the instant invention. The Examples are not intended to represent that the experiments below are all or the only experiments performed.

### Example I

### Generation of CHO Cells Stably Expressing β-Glucocerebrosidase

A plasmid containing a nucleic acid sequence as set forth in SEQ ID NO: 2 (NM_000157.3 available at https://www.ncbi.nlm.nih.gov/nuccore/NM_000157.3) for encoding a β-glucocerebrosidase registered in Genbank with Accession No. NP_000148.2 (https://www.ncbi.nlm.nih.gov/protein/NP_000148.2, SEQ ID NO: 1) and two different antibiotics respectively were transfected into CHO-K1 cells cultured in a BM001H medium purchased from Thermo Fisher Scientific Inc. by a commercially available lipofectin.

Enzyme activity was measured using p-nitrophenyl-b-D-glucopyranoside (Sigma-N7006) as a substrate. The released product was 4-Nitrophenol (Sigma-35836-1G) which was detected by measuring the absorbance at 405 nm using a plate reader (MOLECULAR DEVICE, i3X). A reference standard curve was assayed in parallel to quantitate concentrations of 4-Nitrophenol. Enzyme activity is defined as the amount of enzyme that catalyzes 1 nanomole of 4-Nitrophenol released from p-nitrophenol β-D-glucopyranoside per mL per hour at 37° C.

The stable pool with relatively higher enzyme activity and good viability in batch refeed culture was selected and used for cloning. The results are shown in Table 1.

TABLE 1
Stable Pool Batch Refeed Culture Screening

| | | Peak VCD | | |
|---|---|---|---|---|
| | Avg. Enzyme Activity | (10⁶ | End VCD | End VIA |
| Pool ID | (D8 - end) (U/mL) | cells/mL) | (10⁶ cells/mL) | (%) |
| Top pool 1 | 0.31 | 28.70 | 23.62 | 94.73 |
| Top pool 2 | 0.49 | 26.49 | 23.51 | 98.16 |
| Top pool 3 | 0.18 | 29.29 | 25.51 | 99.41 |
| Top pool 4 | 0.11 | 28.03 | 25.21 | 99.47 |

### Example II

### Mutation of MGAT1 Genes in the Pools Stably Expressing β-Glucocerebrosidase

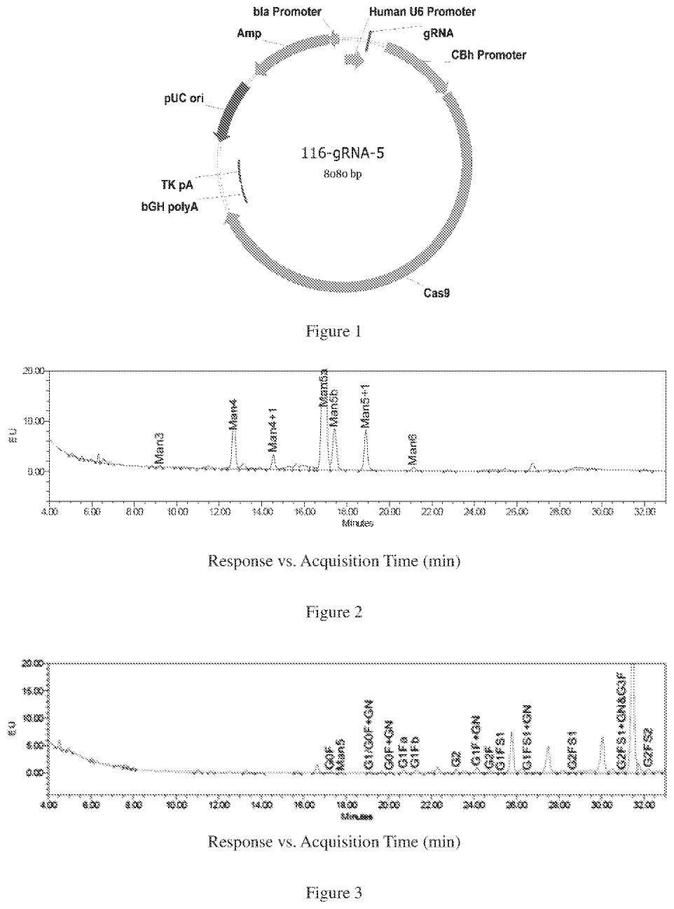

The plasmid containing Cas9 and gRNA targeting MGAT1 was constructed. The gRNA targeting MGAT1 was designed and then ligated into the in house vector which contains Cas9 protein shown as FIG. 1. sgRNA_5 as set forth in SEQ ID NO: 4 (sgRNA_5, TGACAATGGCAAGGAGCAGA) was picked for further use.

The sgRNA_5 plasmid, as shown in FIG. 1, were transfected by a commercially available lipofection into the pool prepared by the method in Example I.

RCAI (Ricinus communis agglutinin) was also applied in clone screening as ricinus communis agglutinin was found to be highly toxic to the wild-type CHO-K1 cells and all the mutants that survived from RCAI selection contained a dysfunctional MGAT1. Once clones recovered in the 96-well plates, they were divided into two plates, while one plate A was cultured in regular culture medium and the other plate B was cultured in medium with RCA selection. Clones that recovered in plate B of the medium with RCAI were selected and screened by enzyme activity assay. After enzyme activity screening, monoclones with relatively higher enzyme activity and verified monoclonality based on their single cell images were selected from the plate A cultured in regular culture medium and then expanded from plate A.

The monoclones were sequentially expanded from 96-well plates into 24-well plates and then spin tubes. Clones with poor growth during expansion were discarded. In the meantime, MGAT1 mutation analysis was conducted for all monoclones by sequencing, and clones with MGAT1 mutation were selected for batch refeed screening. Upon inoculation, cell cultures were seeded by dilution into fresh production medium at a density of 5×10⁵ cells/mL in spin tube. Then majority of medium was refreshed daily by fresh production medium since day 3. When VCD reached plateau, the bleeding process was conducted daily, in which, VCD was adjusted so that it could remain peak VCD in the following day. Glucose was also added into the cultures according to the consumption. Since majority of medium was refreshed frequently, the cells are able to be cultured in an optimal status so that VIA is usually better maintained comparing to traditional fed-batch process. As a result, batch reefed is more suitable to produce fragile biologics such as fusion protein prone to endogenous protease or enzyme in this case. The batch refeed supernatant of clones were subjected for enzyme activity analysis. Enzyme activity of clones was also measured using p-nitrophenyl-b-D-glucopyranoside (Sigma-N7006) as a substrate. The released product was 4-Nitrophenol (Sigma-35836-1G) which was detected by measuring the absorbance at 405 nm using a plate reader (MOLECULAR DEVICE, i3X). A reference standard curve was assayed in parallel to quantitate concentrations of 4-Nitrophenol. Enzyme activity is defined as the amount of enzyme that catalyzes 1 nanomole of 4-Nitrophenol released from p-nitrophenol β-D-glucopyranoside per mL per hour at 37° C. The clone prepared according to the method disclosed in Chinese patent application CN108588127A was used as control.

Top clones with higher β-glucocerebrosidase enzyme activity and Qp were selected and their supernatant was purified by Cation Exchange Chromatography and Hydrophobic Interaction Chromatography. The purified enzyme was subjected for SEC, Caliper-SDS, N-glycan analysis.

Final clone was selected based on enzyme activity, Qp, cell culture performance, monoclonality, low copy number and product quality including SEC, mannose level, Caliper-SDS (non-reduced). Final clone has shown a single nucleic acid insertion into functional area of original MGAT1 sequence which resulted in a malfunction of this gene (Table 2).

TABLE 2
Sequencing Result of Final Clone

MGAT1 DNA Theoretical Sequence:
ATGCTGAAGAAGCAGTCTGCAGGGCTTGTGCTTTGGGGTGCTATCCTCTTTGTGGGCTGGAA
TGCCCTGCTGCTCCTCTTCTTCTGGACACGCCCAGCCCCTGGCAGGCCCCCCTCAGATAGTG
CTATCGATGATGACCCTGCCAGCCTCACCCGTGAGGTGTTCCGCCTGGCTGAGGACGCTGA
GGTGGAGTTGGAGCGGCAGCGGGGGCTGTTGCAGCAAATCAGGGAGCATCATGCTTTGTGG
AGACAGAGGTGGAAAGTGCCCACCGTGGCCCCTCCAGCCTGGCCCCGTGTGCCTGCGACC
CCCTCACCAGCCGTGATCCCCATCCTGGTCATTGCCTGTGACCGCAGCACTGTCCGGCGCTG
CTTGGATAAGTTGTTGCACTATCGGCCCTCAGCTGAGCATTTCCCCATCATTGTCAGCCAGGA
CTGCGGGCACGAGGAGACAGCACAGGTCATTGCTTCCTATGGCAGTGCAGTCACACACATC

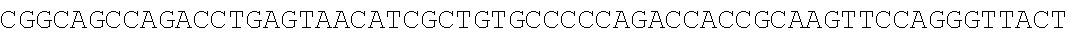

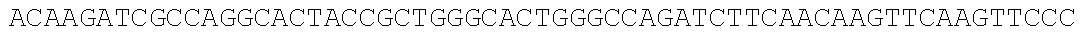

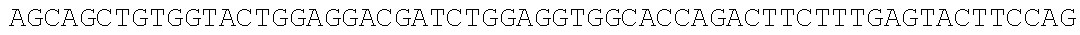

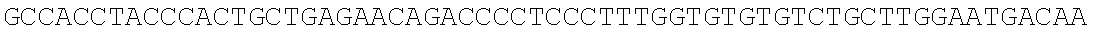

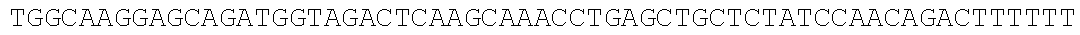

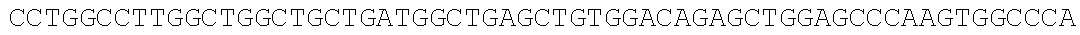

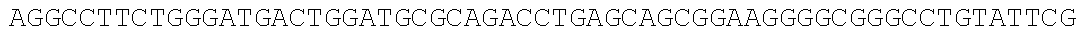

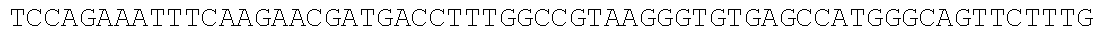

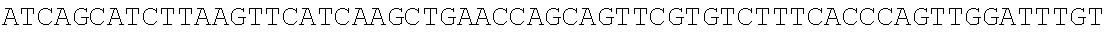

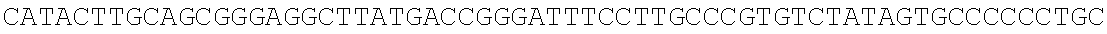

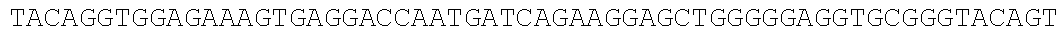

ACACTAGCAGAGACAGCTTCAAGGCCTTTGCTAAGGCCCTGGGTGTCATGGATGACCTCAA
GTCTGGTGTCCCCAGAGCTGGCTACCGGGGCGTTGTCACTTTCCAGTTCAGGGGTCGACGT
GTCCACCTGGCACCCCCACAAACCTGGGAAGGCTATGATCCTAGCTGGAATTAG
MGAT1 Mutation Sequence in Final Clone: Single G inserted into MGAT1
(SEQ ID NO: 3)

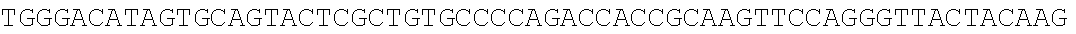

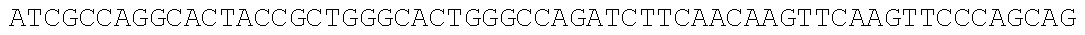

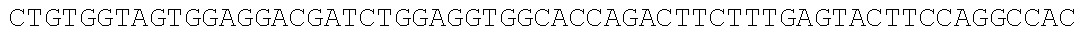

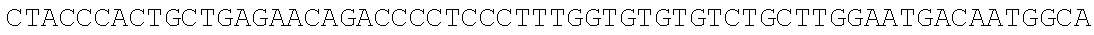

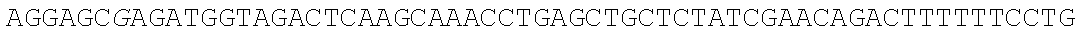

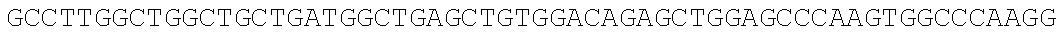

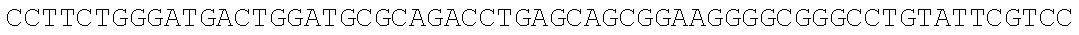

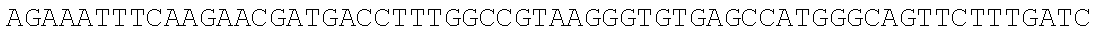

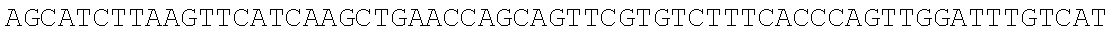

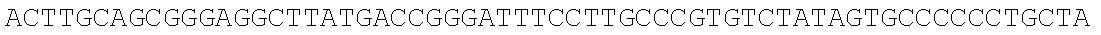

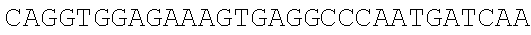

The final clone exhibits higher enzyme activity and better productivity than the control clone (Table 3). The average enzyme activity of final clone is 0.54 U/mL, while the average enzyme activity from control clone is 0.26 U/mL. And the average Qp of final clone is 0.020 U/mL/cell/Day, which is higher than that of control clone.

TABLE 3
Final Clone Performance in Batch Refeeding Screening

| | Avg. Enzyme | Average Qp | Peak | End VCD | End |
|---|---|---|---|---|---|
| | Activity | (U/mL/cell/ | VCD | (10º | VIA |
| Sample ID | (U/mL) | Day) | cells/ml) | cells/ml.) | (%) |
| Control Clone | 0.26 | 0.009 | 35.47 | 31.25 | 99.40 |
| Final Clone | 0,54 | 0.020 | 26.82 | 25.35 | 99.29 |

The Non-Reduced and Reduced purity of β-glucocerebrosidase produced in final clone was detected by Caliper-SDS assay, respectively, Caliper LC90 CE-SDS gel technology, PerkinElmer(®) (Caliper) has designed an automated chip-based fluorescence detection method capable of analyzing proteins in minutes with sensitivity similar to standard SDS-PAGE. The data showed that the purity of Non-Reduced β-glucocerebrosidase is 87.7%, while that of control clone is 70.6% (Table 4).

TABLE 4

| Caliper-SDS Results of Final Clone | | |
|---|---|---|
| | Non-Reduced | Reduced |
| Sample ID | Purity (%) | Purity (%) |
| Control Clone | 70.6 | 63.5 |
| Final Clone | 87.7 | 79.0 |

To analyze monomer and aggregates of final clone, a specific SEC method was developed. Agilent AdvanceBio SEC column (300 Å, 7.8 mm×300 mm, 2.7 μm) was used, and isocratic elution gradient was applied for separation. As shown in Table 5, the percentage of expressed β-glucocerebrosidase monomer from final clone is higher than that in control clone.

TABLE 5
SEC Results of Final Clone

| | SEC-HPLC | | |
|---|---|---|---|
| Sample ID | Main Peak% | HMW Peak% | LMW Peak% |
| Control Clone | 67.7 | 29.5 | 2.8 |
| Final Clone | 81,0 | 19.0 | ND |

Influence of mutation of MGAT1 gene on N-linked glycosylation of β-glucocerebrosidase was determined. N_Glycan profiling was analyzed by UPLC method. The N_oligosaccharide was released by Rapid PNGase F enzyme, then released oligosaccharide was labelled by 2-AB. HILIC based UPLC was applied for separating and quantifying N_glycan. A cell pool with wild type MGAT1 gene is used as control.

As shown in Table 6 and FIG. 2, among all the N-glycans attached to the expressed β-glucocerebrosidase from final clone, 89.3% of them were mannose-terminated N-glycans, i.e. the N-linked Man3, Man4, Man4+1F, Man5, Man5+1F, Man6, etc. Compared with the control pool (FIG. 3), the proportion of N-linked Man5 is significantly increased in the Final Clone, leading to a much higher Man level. These data clearly indicated that mutation of MGAT1 gene in CHO cell expressing β-glucocerebrosidase significantly increased the proportion of the mannose-terminated N-glycans attached to the recombinant β-glucocerebrosidase.

TABLE 6
Glycan Results of Final Clone

| | | | | | | | | Man |
|---|---|---|---|---|---|---|---|---|
| Sample | Man3 | | Man4 ± | \| Man5 | Man5 ± | Man6 | Others | Level |
| ID | (%) | Man4 | IF (%) | (%) | IF (%) | (%) | (%) | (%) |
| Control | | | | 0.1 | | | 99.9 | |
| Pool | | | | | | | | |
| Final | 0.2 | 6.9 | 1.5 | 75.8 | 4.4 | 0.5 | 10.8 | 89.3 |
| Clone | | | | | | | | |

### Example III

### Preparation of the Formulation

β-glucocerebrosidase produced from final clone of Example II is formulated as Table 7.

TABLE 7
Pharmaceutical formulation of ß-glucocerebrosidase

| Material | Function | Concentration |
|---|---|---|
| B-glucocerebrosidase | API | 100 U/mL |
| Citric Acid Monohydrate | Buffer | 0.66 g/L |
| Tri-Sodium Citrate | Buffer | 4.96 g/L |
| Dihydrate | | |
| Sucrose | Osmoregulator | 80.00 g/L |
| Polysorbate 80 (PS80) | Surfactant | 0.20 g/L |
| Water for injection | solvent | adjust to IL |

Note: pH is 6.0 + 0.5 (25 + 2°C), the density is 1.03 g/mL}
(20 + 2° C.).

Material which are used in the formulation include: 20 mM Citrate buffer, 8% (w/v) Sucrose, 0.02% (w/v) PS80, pH 6.0.

1 kg PS80 Stock Solution (10% w/w) Preparation

Ingredients are weighed as follows: 100 g PS80 and 900 g water. The weighed materials are put into a container. Mix the solution until all the PS 80 is visually dissolved.

### 1 kg Formulation Buffer Preparation

Ingredients are weighed as follows: Citric Acid Monohydrate 0.64 g, Tri-Sodium Citrate Dihydrate 4.81 g, Sucrose 77.52 g, 10%(w/w) PS80 stock solution 1.94 g, water 915.09 g. The weighed excipients are all transferred into a 1 L container. Water is added to the container to a total weight of 1000 g. The solution is mixed until all the excipients are completely disolved by visual.

### 1 L DS Dilution and Mixing

Formulation buffer will be added into the DS (e.g. the specific enzyme activity is 44.5 U/mg and the enzyme activity is 4.1 mg/mL) to complete the protein (100U/mL) compounding. 0.55 L DS is poured into a container, and then 0.45 L formulation buffer is put into the same container, too. The diluted DS is then homogenously mixed.

### Example IV

### Formulation Studies

Table 8 shows key performance studies of the formulation. The studies demonstrates the formulation is stable at 2-8° C. for at least 12 weeks or at room temperature (about 25° C.) for at least 12 weeks or at 40° C. for at least 4 weeks. The formulation retains its physical and/or chemical and/or biological activity upon storage under different conditions.

TABLE 8A
Appearance/Osmolality/pH/Moisture/Reconstitution time of the Formulation

| | | Appearance | | | | | |
|---|---|---|---|---|---|---|---|
| | Sampling | Before | After | Osmolality | | | Reconstitution |
| Condition | point | reconstitution | reconstitution | mOSm/kg | pH | Moisture | time |
| TO | TO | White cake | Colorless, | 317 | 6.0 | 1.5 | 47s |
| 5 ± 3 ºC. | 4 weeks | without | Clear, No | No detection | 6.0 | 1.5 | 37s |
| | 12 weeks | shrinkage | visible | No detection | 5.9 | 1.4 | 41s |
| | | | particles | | | | |
| 25 ± 2 °C., | 4 weeks | | | No detection | 6.0 | 1.5 | 38s |
| 60 ± 5% RH | 12 weeks | | | No detection | 5.9 | 1.8 | 41s |
| 40 ± 2 ºC. | 2 weeks | | | No detection | 5.9 | 1.5 | 1min25s |
| 75 ± 5% RH | 4 weeks | | | No detection | 5.9 | 1.8 | 40s |

TABLE 8B
Protein concentration/Purity of the Formulation

| | | | Purity % | | | | | |
|---|---|---|---|---|---|---|---|---|
| | | | | | | | High | Low |
| | | | | | | | moleular | moleular |
| | | Protein | CE-SDS(R) | | | SEC | weight | weight |
| | Sampling | concentration | % light chain ± | CE-SD(NR) | RP-HP LC | Main | specie | species |
| Condition | point | mg/mL | % heavy chain | % purity | purity % | peak % | 5% | % |
| TO | TO | 2.5 | 92.2 | 98.4 | 93.8 | 98.6 | 1.4 | Not |
| | | | | | | | | detected |
| 3ºC | 4 weeks | 2.5 | 92.0 | 98.4 | 92.5 | 98.2 | 1.8 | Not |
| | | | | | | | | detected |
| | 12 weeks | 2.5 | No | 97.4 | 91.6 | 98.5 | 1.5 | Not |
| | | | detection | | | | | detected |
| 25 ± 2 ° C., | 4 weeks | 2.5 | 92.0 | 98.2 | 90.9 | 98.1 | \| 1.9 | Not |
| | | | | | | | | detected |
| 60 ± 5% RH | 12 weeks | 2.5 | No | 97.6 | 89.7 | 98.4 | 1.6 | Not |
| | | | detection | | | | | detected |
| 40 ± 2 °C., | 2 weeks | 2.5 | 92.1 | 98.6 | 89.0 | 98.7 | 1.3 | Not |
| | | | | | | | | detected |
| 75 ± 5% RH | 4 weeks | 2.5 | 91.7 | 98.1 | 88.0 | 98.2 | 1.8 | Not |
| | | | | | | | | detected |

TABLE 8C
DLS/HIAC/Enzyme activity of the Formulation

| | Sampling | DLS | | HIAC | | | Enzyme activity |
|---|---|---|---|---|---|---|---|
| Condition | point | Z-average | PdI | ≥2 um | ≥10 um | ≥25 um | U/mL |
| TO | TO | 12.3 | 0.3 | 1002 | 47 | 3 | 106.7 |
| 5 ± 3ºC | 4 weeks | 12.4 | 0.3 | 541 | 20 | 1 | 82.7 |
| | 12 weeks | 12.3 | 0.3 | 592 | 13 | 0 | 96.9 |
| 25 ± 2 ° C., | 4 weeks | 12.7 | 0.3 | 1433 | 67 | 2 | 91.3 |
| 60 ± 5% RH | 12 weeks | 13.1 | 0.3 | 1618 | 13 | 2 | 92.5 |
| 40 ± 2°C, | 2 weeks | 12.5 | 0.3 | 485 | 24 | 0 | 91.0 |
| 75 ± 5%RH | 4 weeks | 12.6 | 0.3 | 688 | 43 | 2 | 88.6 |

### Appearance

The outer wall of the glass vials is cleaned, and then the neck of glass vials is held closed to the edge of the gobo of YB-2 lightbox at a distance of 25 cm away. The appearance of samples, including color, clarity and visible particles, is examined against black and white background with an illumination level at 2000-3750 lx.

### Osmolality

The osmolality of 20 μL undiluted samples is measured by Advanced 2020. Before and after the measurement, the osmometer is calibrated by 290 mOsm reference solution.

pH

The pH meter is calibrated with three different standard buffers (pH 4.01, 7.00 and 9.21) prior to use. After that, pH is measured at a loading volume of 50 μL for each sample.

### Moisture

The measurement of moisture content is performed using Mettler Toledo C30D Karl Fischer coulometer. The room temperature is about 15-30° C., and the air humidity is below 50%. The analyte is added first, and the instrument is balanced until the relative shift is below 15 μg/min. Then the lyophilized powder is opened and weighed, and added to the instrument until the instrument shows the moisture content reading. In order to avoid the lyophilized power absorbing water from the air, the process must be conducted quickly from opening to weighing the power.

### Reconstitution Time

The flip-off aluminum caps are removed from glass vials, the ultrapure water is added into the vials from the vial wall with sterile injectors to avoid the direct impaction against lyophilized samples. The vials are rotated slightly, and are made static after being infiltrated by ultrapure water completely. The time is recorded when the ultrapure water is injected until the lyophilized samples are fully dissolved.

### Protein Concentration

After the samples are mixed evenly, protein concentration is determined by UV280 readings using a NanoDrop 2000 spectrophotometer. The extinction coefficient is 1.703 AU*mL*mg−1*cm−1. All measurements are repeated twice at a loading volume of 2.5 μL each time and an average is taken.

### CE-SDS(NR&R)

CE-SDS, driven by a high-voltage direct current electric field, is a capillary electrophoresis method that allows sample separation by molecule size. The medium is a continuous gel filled in the capillary which forms a molecular sieve in the capillary and serves as a separation channel. For CE-SDS, the sample preparation before electrophoresis involves heat denaturing of a specified concentration of sample in the presence of SDS, which masks the samples intrinsic charge and confers all the species similar charge-to-size ratios. Upon application of a constant electric field, the samples migrate towards the anode with different speeds depending on their size.

### CE-SDS-NR

CE_NR method for WBP108 is carried out under the following experimental conditions: 50 μg of sample is transferred into the dilution solution PB-CA, making a total volume of 25 μL, then 75 μL of 1% SDS Sample Buffer and 5 μL alkylation reagent are added to make a final volume of 105 μL, and the solution is mixed thoroughly. The sample is incubated in heating block for 10 minutes at 60° C., and then is cooled down at room temperature for at least 3 minutes. 90 μL of the prepared sample is transferred into the insert and the inserts are placed into the vials. The vials are capped, and then placed into the sample vial holder for sample analysis, and the injection time is 40 seconds.

### CE-SDS-R

CE_R method is carried out under the following experimental conditions: 50 μg of sample is transferred into the dilution solution PB-CA, making a total volume of 25 μL, then 75 μL of 1% SDS Sample Buffer and 5 μL reduced reagent are added to make a final volume of 105 μL, and the solution is mixed thoroughly. The sample is incubated in heating block for 10 minutes at 60° C., and then is cooled down the samples at room temperature for at least 3 minutes. Transfer 90 μL of the prepared sample is transferred into the insert and place the inserts are placed into the vials. Cap The vials are capped, and then placed them into the sample vial holder for sample analysis, and the injection time is 40 seconds.

### RP-HPLC

Reversed-phase chromatography (RPC) refers to elution chromatography that uses non-polar reversed-phase media as the stationary phase and an aqueous solution of a polar organic solvent as the mobile phase to separate and purify solutes based on difference in solute polarity (hydrophobicity). The solute is distributed on the surface of the stationary phase through hydrophobic interactions. However, the surface of the RPC stationary phase is completely covered by non-polar groups, which shows strong hydrophobicity. Therefore, it is necessary to use a polar organic solvent (such as methanol, acetonitrile, etc.) or its aqueous solution for elution and separation.

RP method is carried out on a HPLC system under the following experimental conditions: Waters BioResolve TM PR mAb Polyphenyl, 450A, 2.7 um,2.1 mm*150 mm, 1/pk is used as the column; 0.1% TFA in H20 and 0.1% TFA in ACN are applied as mobile phase; 280 nm is chosen as UV detector wavelengths; column temperature is set at 45° C.; samples are settled at 5° C.; isocratic flow rate is 0.3 mL/min; the injection quantity is 10 μg and the running time is 27 min.

### SEC-HPLC

The method of Size Exclusive Chromatography (SEC), also known as gel filtration, separates molecules by difference in size (hydrodynamic radii) as they pass through a SEC resin packed in a column. SEC resins consist of a porous matrix of spherical particles that is designed not to interact with the molecules to be separated. After being applied to the column, molecules that are larger than the pores are unable to diffuse into the beads, so they elute first. Molecules that are smaller than the pore size can penetrate the pores to various degrees based on their size.

SEC method is carried out on a HPLC system under the following experimental conditions: Agilent AdvanceBio SEC (300 Å, 7.8 mm*300 mm, 2.7 um) is used as the column; 55 mM Na-Citrate+200 mM L-Arginine, pH 5.5±0.1 is applied as mobile phase; 280 nm is chosen as UV detector wavelengths; column temperature is set at 25° C.; samples are settled at 5° C.; isocratic flow rate is 0.6 mL/min; the injection quantity is 30 μg and the running time is 40 min. This SEC method is applied for all studies in this report with the exception noted below.

### DLS

Scattering phenomenon would occur when laser beam passes though the particles due to Brownian motion of particles in solution. The hydrodynamic radius of particles could be calculated and obtained by measuring the diffusion coefficient of particles in solution by DLS. The DLS analysis was performed on a Malvern ZEN3600. 70 μL sample was added into a disposable cuvette in the biosafety hood for testing at 25° C.

### HIAC

Sub-visible particles are monitored by a HIAC system. Each sample is tested for four consecutive runs (1 mL/run). The first run result is discarded and the average value of the rest three runs is recorded. At the end, data is auto calculated by the software and the results are presented as average numbers of particles ≥10 μm and ≥25 μm per mL.

### Enzyme Activity

Diluted enzyme is mixed with 20 mM p-NPG at 37° C. for 1 h. 1 M Glysine is added to stop the reaction. The absorbance at 405 nm is measured, producing a signal that is proportional to the acitivity of enzyme in the sample. Plot standard curve of enzyme reaction product P-NP and regress according to a linear regression model using the SoftMax Software. The enzyme activity of testing samples is calculated by comparing the OD value of testing samples to the standard curve.

Those skilled in the art will further appreciate that the present disclosure may be embodied in other specific forms without departing from the spirit or central attributes thereof. In that the foregoing description of the present disclosure discloses only exemplary embodiments thereof, it is to be understood that other variations are contemplated as being within the scope of the present disclosure. Accordingly, the present disclosure is not limited to the particular embodiments that have been described in detail herein. Rather, reference should be made to the appended claims as indicative of the scope and content of the present disclosure.

1. A genetically modified cell line deficient in mannosyl (alpha-1,3-)-recombinant glycoprotein beta-1,2-N-acetylglucosaminyltransferase 1 (MGAT1) gene, comprising an insertion of less than 600 bp in the coding region of a chromosomal sequence encoding MGAT1.

2. The genetically modified cell line of claim 1, wherein the cell line comprises an insertion of less than 400, 200, 100, 50, 30, 10, or 5 bp in the coding region of a chromosomal sequence encoding MGAT1.

3. The genetically modified cell line of claim 1, wherein the cell line comprises an insertion of 1 bp in the coding region of a chromosomal sequence encoding MGAT1.

4. The genetically modified cell line of claim 1, wherein the chromosomal sequence encoding MGAT1 is modified using a targeting endonuclease-mediated genome editing technology.

5. The genetically modified cell line of claim 4, wherein the targeting endonuclease-mediated genome editing technology is a Clustered Regularly Interspaced Short Palindromic Repeats (CRISPR) , Zinc Finger nuclease (ZFN) or Transcription activator-like effector nuclease (TALEN).

6. The genetically modified cell line of claim 1, wherein the cell is an animal cell, preferably a mammalian cell, further preferably a Chinese Hamster Ovary (CHO) cell.

7. The genetically modified cell line of claim 1, wherein the cell line expresses at least one glycoprotein comprising one or more terminal mannose residues.

8. The genetically modified cell line of claim 7, wherein the glycoprotein is an enzyme.

9. A method for producing a recombinant glycoprotein, comprising
(1) culturing the cell line of claim 7; and
(2) recovering the glycoprotein.

10. A method for producing a recombinant glycoprotein comprising:
(1) introducing a gene encoding the recombinant protein into a cell line;
(2) mutating mannosyl (alpha-1,3-)-recombinant glycoprotein beta-1,2-N-acetylglucosaminyltransferase 1 (MGAT1) gene in the cell line obtained by (1); and
(3) expressing the recombinant glycoprotein;
wherein, the MGAT1 gene is mutated by CRISPR, in which sgRNA comprises or consists of a nucleic acid sequence of SEQ ID NO: 4, or comprises or consists of a nucleic acid sequence having at least 90%, 95%, 96%, 97%, 98% or 99% sequence identity to SEQ ID NO: 4.

11. The method of claim 10, wherein the cell line is selected with RCA.

12. The method of any of claim 10, wherein the recombinant glycoprotein is an enzyme.

13. The method of claim 12, further comprising selecting the mutated cell line in a batch refeeding assay.

14. The method of claim 13, wherein the batch refeeding assay comprises refreshing the production medium daily.

15. The method of any of claim 10, wherein the recombinant glycoprotein has a N-glycan containing one or more terminal mannose residues and optionally 1 to 3 fucose residues which do not block the terminal mannose residues, wherein the N-glycan is selected from the group consisting of Man3, Man4, Man4+1F, Man5, Man5+1F, and Man6.

16. The method of claim 15, wherein the proportion of the mannose-terminated N-glycans attached to the recombinant β-glucocerebrosidase is more than 80%, 85%, 86%, 87%, 88%, or 89%.

17. The method of claim 11, wherein the enzyme activity of the recombinant glycoprotein is more than 0.5 U/mL

18. The method of claim 11, wherein the cell is an animal cell.

19. A recombinant glycoprotein. produced by the cell line of claim 1.

20. A method for treating a β-glucocerebrosidase-deficiency-related disease comprising administering the recombinant glycoprotein of claim 19 to a subject in need thereof.

21. A cell, clone or cell line generated by the method claim 9.

22. A pharmaceutical formulation comprising a therapeutically effective amount of a recombinant enzyme, a buffer, an osmoregulator and a surfactant.

23. The formulation of claim 22, wherein the enzyme is β-glucocerebrosidase present at an amount of 50-150 U/ml.

24. The formulation of claim 22, wherein the buffer is a citrate buffer present at an amount of 2-10 g/mL.

25. The formulation of claim 22, wherein the osmoregulator is sucrose present at amount of 40-120 g/L.

26. The formulation of claim 22, wherein the surfactant is polysorbate 80 present at an amount of 0.1-1 g/L.

27. The formulation of claim 22, wherein the formulation has a pH of 5-7.


SEQUENCE LISTING (4 sequences)

SEQ ID NO: 1 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 2 - DNA, 2324 bp, Homo sapiens
atcacatgacccatccacatcgggaagccggaattacttgcagggctaacctagtgcctatagctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagcccagtca

SEQ ID NO: 3 - DNA, 1344 bp, Homo sapiens
atgctgaagaagcagtctgcagggcttgtgctttggggtgctatcctctttgtgggctggaatgccctgctgctcctcttcttctggacacgcccagcccctggcaggcccccctcagatagtgctatcgatgatgaccctgccagcctcacccgtgaggtgttccgcctggctgaggacgctgaggtggagttggagcggcagcgggggctgttgcagcaaatcagggagcatcatgctttgtggagacagaggtggaaagtgcccaccgtggcccctccagcctggccccgtgtgcctgcgaccccctcaccagccgtgatccccatcctggtcattgcctgtgaccgcagcactgtccggcgctgcttggataagttgttgcactatcggccctcagctgagcatttccccatcattgtcagccaggactgcgggcacgaggagacagcacaggtcattgcttcctatggcagtgcagtcacacacatccggcagccagacctgagtaacatcgctgtgcccccagaccaccgcaagttccagggttactacaagatcgccaggcactaccgctgggcactgggccagatcttcaacaagttcaagttcccagcagctgtggtagtggaggacgatctggaggtggcaccagacttctttgagtacttccaggccacctacccactgctgagaacagacccctccctttggtgtgtgtctgcttggaatgacaatggcaaggagcagatggtagactcaagcaaacctgagctgctctatcgaacagacttttttcctggccttggctggctgctgatggctgagctgtggacagagctggagcccaagtggcccaaggccttctgggatgactggatgcgcagacctgagcagcggaaggggcgggcctgtattcgtccagaaatttcaagaacgatgacctttggccgtaagggtgtgagccatgggcagttctttgatcagcatcttaagttcatcaagctgaaccagcagttcgtgtctttcacccagttggatttgtcatacttgcagcgggaggcttatgaccgggatttccttgcccgtgtctatagtgcccccctgctacaggtggagaaagtgaggaccaatgatcagaaggagctgggggaggtgcgggtacagtacactagcagagacagcttcaaggcctttgctaaggccctgggtgtcatggatgacctcaagtctggtgtccccagagctggctaccggggcgttgtcactttccagttcaggggtcgacgtgtccacctggcacccccacaaacctgggaaggctatgatcctagctggaattag

SEQ ID NO: 4 - DNA, 20 bp, Artificial Sequence
  sgRNA_5
tgacaatggcaaggagcaga